(19) United States
(12) Patent Application Publication
(10) Pub. No.: US 2004/0157809 A1
(43) Pub. Date: Aug. 12, 2004

(54) Composition comprising an androgen receptor blocker and an insulin sensitizing agent and use thereof for treatment of polycystic ovary syndrome

(76) Inventors: Francis De Zegher, Tervuren (BE); Lourdes Ibanez, Barcelona (ES)

Correspondence Address: CLARK & ELBING LLP, 101 FEDERAL STREET, BOSTON, MA, 02110, US

(21) Appl. No.: 10/474,539

(22) PCT Filed: Apr. 9, 2002
(86) PCT No.: PCT/BE02/00052

(30) Foreign Application Priority Data
Apr. 10, 2001 (GB) 0108863.2

(51) Int. Cl. A61K 31/56; A61K 31/155

(52) U.S. Cl. 514/171; 514/635

(57) ABSTRACT

Novels compositions comprising an endrogen receptor blocker and an insulin sensitizing agent are presented. The use of these compositions for the treatment of persons with an endocrine-metabolic disorder such as polycystic ovary syndrome is disclosed. The novel compositions are able to reduce the levels of triglycerides and male hormones. The novel compositions enables the reduction of preferentially abdominal fat resulting in a change in body composition.

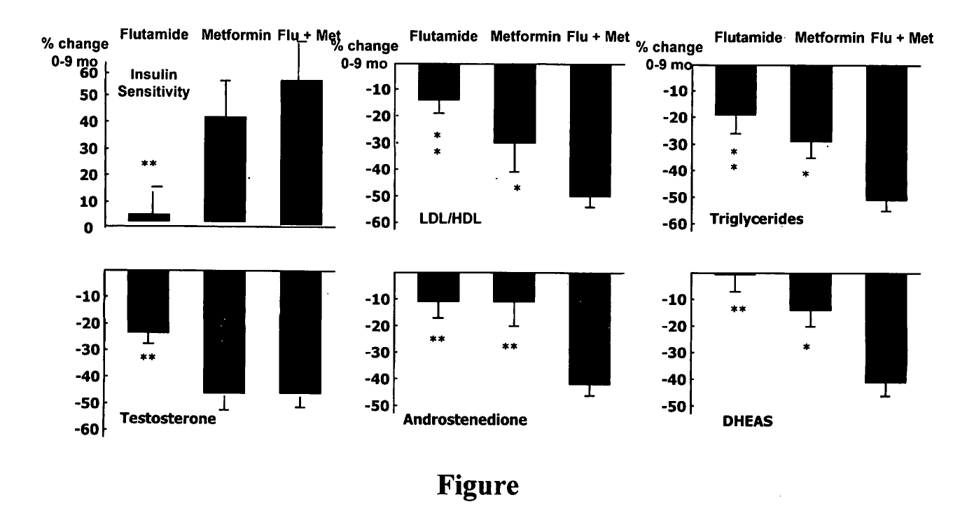

### FIELD OF THE INVENTION

The present invention relates to normalizing the endocrine-metabolic status of female human subjects with the endocrine-metabolic disorder polycystic ovary syndrome (PCOS) and/or to reduce the amount of total body fat, especially by reducing the amount of abdominal fat, in said subjects. The reduction in abdominal fat results in a decreased risk for cardiovascular disorders.

### BACKGROUND OF THE INVENTION

Of the several endocrine disorders, Polycystic Ovary Syndrome (PCOS) is considered to be the most common reproductive endocrine disorder in women. The endocrine-metabolic hallmarks of PCOS in women are hyperinsulinism, hyperandrogenism and dyslipidemia (Taylor in (1998) Endocrinol Metab Clin North Am. 27:877-902; Rosenfield in (1997) Baillière's Clin Obstet Gynaecol. 11:307-333; Dunaif in (1997) Endocr Rev. 18:774-800; Robinson et al. (1996) Clin Endocrinol. 44:277-284)

Insulin-sensitizing and anti-androgen monotherapies are partially effective treatments that act through different pathways and, accordingly, have a different spectrum of endocrine-metabolic actions (Simard et al. (1986) Mol Cell Endocrinol. 44:261-270; Bailey et al. (1996) N Engl J Med 334:574-579; Schoonjans and Auwerx (2000) Lancet. 355:1008-1010.).

Treatment with flutamide, a non-steroidal anti-androgen of the androgen receptor blocker type, reduces hirsutism and circulating levels of androgens, triglycerides and low-density lipoprotein (LDL) cholesterol in adolescents and women with PCOS, but does not restore menstrual cyclicity. In addition, it fails to increase high-density lipoprotein (HDL) cholesterol or to decrease hyperinsulinemia, i.e. to affect two major risk factors for subsequent cardiovascular disease (Diamanti-Kandarakiset et al. (1998) J Clin Endocrinol Metab. 83:2699-2705; de Leo et al. (1998) J Clin Endocrinol Metab. 83:99-102; Ibáñez et al. (2000a) J Clin Endocrinol Metab. 85:3251-3255; Mather et al. (2000) Fertil Steril. 73:150-156; Goldbourt et al. (1997) Arterioscler Thromb Vasc Biol. 17:107-113.).

Insulin-sensitizing treatment with e.g. metformin is known to reduce the hyperinsulinism, the hyperandrogenism and the atherogenicity of the lipid profile, and to restore eumenorrhea and ovulatory function, but it is less effective in decreasing hirsutism (Velázquez et al. (1994) Metabolism. 43:647-654; Nestler and Jakubowicz (1996) N Engl J Med. 335:617-623; Nestler and Jakubowicz (1997) J Clin Endocrinol Metab. 82:4075-4079; Moghetti et al. (2000) J Clin Endocrinol Metab. 85:139-146; Ibáñez et al. (2000b) J Clin Endocrinol Metab. 85:3526-3530; Nestler et al. (1998) N Eng J Med. 338:1876-1880; DeLeo et al. (1999) Fertil Steril. 72:282-285; Hasegawa et al. (1999) Fertil Steril. 71:323-327).

Central or android patterns of fat distribution in females have been linked to the effects of excess androgens (Lovejoy et al. (1996) J Clin Endocrinol Metab. 81:2198-2203; Elbers et al. (1997) J Clin Endocrinol Metab. 82:2044-2047; Nilsson et al. (1998). J Clin Invest. 101:74-78). Increased body fat and central adiposity have also been reported in girls and women with hyperinsulinemic-hyperandrogenemic states, such as precocious pubarche and polycystic ovary syndrome (PCOS) (Douchi et al. (1995) Obstet Gynecol. 86:516-519; Kirchengast & Huber J. (2001) Hum Reprod. 16:1255-1260; Pech et al. Proceedings of the 83rd Annual Meeting of The Endocrine Society, Denver, Colo., 2001; p. 403). This pattern of body composition occurs independently of obesity, and may have long-term implications for cardiovascular disease risk (McFarlaneet al. J Clin Endocrinol Metab. 86:713-718; Vanhalaet al. (1998) Int J Obes. 22:369-374.).

Obesity is a prominent feature of PCOS, at least 50% of patients with PCOS are obese. Obesity exerts an additive synergistic effect on the manifestations of PCOS, independently impacting insulin sensitivity, risk for diabetes and adverse cardiovascular profile.

In obese PCOS women, insulin-sensitizing monotherapy added to a hypocaloric diet induces significant reductions in both body weight and abdominal fat, while decreasing insulin and androgen concentrations (Pasquali et al. (2000) J Clin Endocrinol Metab. 85:2767-2774.). In this study changes in waist-to-hip-ratio (WHR) are similar in PCOS women and controls regardless of the pharmaceutical treatment. The observed changes are small and not sufficient to substantially reduce the risk of obesity associated coronary diseases. Obesity is generally recognized, by organizations as the WHO, as a disorder and not as a cosmetic problem. Furthermore, it is well known that fat distribution is a better predictor for cardiovascular diseases than the degree of obesity. From a population study of 44,000 women a WHR above 0.76 was associated with a more than 2-fold higher risk of coronary heart diseases (Rexrode et al (1998) JAMA 280, 1743-1848).

From the preceding summary is it clear that there is a need for improved therapies which can treat a broader spectrum than the existing monotherapies. Apart from an improved treatment for the metabolic-endrocine aspects, there is a particular need for a reduction in total body fat mass and more particular abdominal fat mass in order to decrease the risk of cardiovascular diseases in persons with a predisposition for cardiovascular diseases such as women with polycystic ovary syndrome.

### SUMMARY OF THE INVENTION

The present invention relates to a combination therapy and pharmaceutical compositions for the treatment of an endocrine-metabolic disorder such as polycystic ovary syndrome in women. The combination therapy of the present invention uses an androgen receptor blocker, an insulin sensitizer and optionally a contraceptive.

In a preferred embodiment of the invention the androgen receptor blocker is flutamide and the insulin sensitizer is metformin. In another embodiment the amount of flutamide being used is substantially lower than being used in PCOS flutamide monotherapies. The combination therapy of the present invention has the surprising and unexpected effect that apart from additive, cumulative and synergistic effects on glycerides and hormone levels, an unprecedented loss of fat, predominantly abdominal fat is encountered with a minimal loss in body weight and an increase in lean weight as well in obese as non obese persons.

Due to this selective reduction in fat, the risk of cardiovascular disease is decreased. The present combination therapy provides apart from the improved endocrine and metabolic improvements a highly appreciated change in body shape by a reduction of the waist to hip ratio.

The present invention will now be described with respect to the attached figure.

### BRIEF DESCRIPTION OF THE DRAWING

The figure shows the changes in insulin sensitivity, LDL/HDL ratio, triglycerides, androstenedione, testosterone and dehydroepiandrosterone sulfate (DHEAS) after 9 months of monotherapy with flutamide (250 mg/day) or metformin (1275 mg/day) or after combination therapy with flutamide (250 mg/day) and metformin (1275 mg/day).

### DEFINITIONS

“abdominal fat” relates to fat located in the abdominal region, The abdominal region is defined as the area encompassed between the dome of the diaphragm (cephalad limit) and the top of the greater throcanter (caudal limit) (Taylor et al. (1998) Am J Clin Nutr. 67:44-49.) “analog” in the present invention refers to compounds having unrelated structure but similar function.

“androgen receptor blocker” is any substance capable of preventing full expression of the biologic effects of androgenic hormones on responsive tissues, by inhibiting androgenic effects by competing for binding sites at the cell surface. Therefore, products with an antagonistic effect on the target tissue such as oestrogens are not androgen receptor blockers within the scope of the present invention. Examples of androgen receptor blockers are flutamide, spirolactone and cyproterone acetate.

“combination therapy” in this invention relates to the administration of different active agents. Within the context of this invention known combination therapies are the administration of an androgen receptor blocker and an contraceptive, an insulin sensitizing agent and an oestrogen. Combination therapy in this invention also relates to the novel combined administration of an androgen receptor blocker and an insulin sensitizing agent, either simultaneously or sequentially.

“combined administration” deals with a way of administration wherein during the therapy two or more agents are used. These agents can be administered at the same moment or with a certain interval (for example before or after a meal, at morning or evening, on alternating day. The two or more active agents can be within the same pharmaceutical or in different pharmaceuticals and can be administered separately or combined. These can be within the same packing material (blister, vial, container) or within different packing materials. Further the different agents can be administered by the same way, wherein oral administration is preferred, but can also be administered by different ways such as administration of one agent by injection and one or more agents orally. As an example we refer to a way of administration wherein the androgen receptor blocker and the insulin sensitizing agent are administered orally on a daily base and the contraceptive is administered by injection on a weekly or monthly base.

“derivative” in the present invention refers to compounds with similar chemical structure and similar function.

“Flutamide” is the generic name of the acetanilid nonsteroidal, orally active anti-androgen with the chemical name 2-methyl-N-[4-nitro-3(trifluoromethyl)phenyl] popanamide. Flutamide is well known for its use in the treatment of prostate cancer and is also known under trade names such as Euflex or Eulexin.

“insulin sensitizing agent” relates to compounds reversing the effects of insulin resistance by a causing a decrease in circulating insulin levels. Examples are metformin, troglitazone, rosiglitazone, pioglitazone.

“lean mass of the body” relates to the mass of the body excluding fat and bones. An increase of lean mass generally applies to an increase in muscle. A reduction of fat without change in body weight is indicative of an increase of the lean mass, mainly by a conversion of fat into muscle.

“Metformin” is the generic name of an oral antihyperglycemic drug used in the emanagement of non-insulin-dependent diabetes mellitus (NIDDM) and having the chemical name N,N-dimethylimidodicarbonimidic diamide hydrochloride metformin is e.g. known under trade names such as Glucophage or Dianben.

“monotherapy” in the present invention relates to the administration of a single active agent for the treatment of PCOS. It relates thus to the administration of an androgen receptor blocker or to the administration of an insulin sensitizing agent. The administration of more than one androgen receptor blocker is also considered to be a monotherapy as is the administration of more than one insulin sensitizing agent.

“PolyCystic Ovary Syndrome” (abbreviated as PCOS) as used herein relates to an endocrine-metabolic disorder in women which is characterized by hyperinsulinism, hyperandrogenism, and usually dyslipidemia and anovulation.

“synergism or synergistic” refers to the coordinated or correlated action of two or more compound or processes so that the combined action is greater than the sum of each acting separately, or the combined action is a action which does not occur when each compound or process or acting separately.

“truncal fat” relates to fat localized in the truncal region. The truncal region is defined as the tissue area bordered by a horizontal line below the chin, vertical borders lateral to the ribs, and oblique lines passing through the femoral necks.

“waist to hip ratio” (abbreviated as WHR) is the ratio between the waist circumference and the hip circumference. Waist circumference is measured at the end of expiration to the nearest millimeter, using a measuring tape placed around the waist at the level of the umbilicus. Hip circumference is also measured to the nearest millimeter, at the level of maximal anteroposterior hip excursion.

### DETAILED DESCRIPTION OF THE INVENTION

The present invention is related to compositions and uses thereof for the treatment of human subjects with metabole-endocrine disorders characterized by hyperinsulinism and hyperandrogenism.

One aspect of the present invention relates to a combination therapy for human female subjects with the metabole-endocrine disorder PCOS. The present invention applies as well to PCOS patients with obesity as to PCOS patients without obesity. The present invention applies as well to young adolescent, adolescent, adult premenopausal and adult postmenopausal women.

The combination therapy of the present invention relates to the use of a combination of an androgen receptor blocker and an insulin sensitizing agent, optionally further combined with a contraceptive for the manufacture of a medicament for the treatment of PCOS women.

The androgen receptor blocker is preferably a non-steroid androgen. Several non-steroid androgens are known such as flutamide, spirolactones or cyproterone acetate. In the present invention, Flutamide is disclosed as a preferred non-steroid androgen receptor blocker. Derivatives, analogs and salts of flutamide are within the scope of the invention. The present invention also relates to the administration of lowered levels of androgen receptor blockers compared with levels used in monotherapies. The concentration is preferably 50% or lower of commonly used concentrations of androgen receptor blockers used for the treatment of PCOS. The concentration is more preferably 25% or lower of commonly used concentrations of androgen receptor blockers used for the treatment of PCOS. For example the daily dose of flutamide is about 250 mg, preferably about 125 mg or less and more preferably about 50 mg. Other doses between about 50 mg and about 125 mg, 125 mg and 250 mg can also be used for the present invention.

Alternative androgen receptor blockers, other than flutamide can be equally suited for the combination therapy of the present invention. Examples of such compounds are cyproterone acetate or spironolactone.

In order to define the concentration of such an alternative androgen receptor blocker, the activity of such compound has to be determined. This can be performed for example by an assay such as an androgen responsive element reported assay (eg. Haelens et al. (2001) Biochem. J. 353,611-620. In this type of assay a vector comprising an androgen responsive element such as the androgen promoter is operably fused to a reporter construct such as green fluorescent protein (GFP) or beta galactosidase or luciferase. Said vectors are transfected into eukaryotic cells expressing androgen receptors. After administration of an androgen, the reporter construct is transcribed and translated which can be assayed by the measuring the fluorescence produced by the GPF, the staining produced by the beta galactosidase after administration of IPTG (isopropylthiogalactoside), the light emission produced by the luciferase after administration of ATP (adenosine triphosphate). With this type of assay, the decrease of androgen response after admixture with a known amount of flutamide is determined. Similarly, the amount of an alternative androgen receptor blocker can be titrated in order to determine the concentration with the same effectivity of inhibition as a concentration of flutamide.

The insulin sensitizing agent can be any suitable insulin sensitizing agent. In the present invention, the insulin sensitizing agent is preferably metformin. However, derivatives, salt and analogs of metformin are also within the scope of the invention. Further examples of insulin sensitizing agents are troglitazone, rosiglitazone and pioglitazone. The present invention discloses the use of 1275 mg Metformin on a daily base, although other doses leading to the effects of the combination therapy are within the scope of the invention. Acceptable doses are between 1700 and 850 mg on a daily basis or between 850 and 425 mg on a daily basis, such as about 1700 mg metformin on a daily basis, about 850 mg on a daily basis or about 425 mg on a daily basis.

When metformin is replaced by another dose of a metformin derivative or analog the amount of the of this compound can be determined by supplying such a dose which results in the same decrease in circulating insulin levels as obtained by the amount of metformin being used. Circulating insulin levels can be determined by standard blood analysis techniques.

In another embodiment the invention relates to combination therapies wherein flutamide and/or metformin are replaced by other androgen receptor blockers or insulin sensitizing agents. Therefore combinations such as flutamide and troglitazone, cyproterone acetate and troglitazone, spirolactone and troglitazone, flutamide and rosiglitazone, cyproterone acetate and rosiglitazone, spirolactone and roziglitazone, flutamide and pioglitazone, cyproterone acetate and pioglitazone, spirolactone and pioglitazone, cyproterone acetate and metformin, spirolactone and metformin, are within the scope of the combination therapy of the present invention.

The combination therapy of the present invention can be administered in several ways. The frequency of the administration is preferably on a daily base, although other clinically acceptable regimens are not excluded. The androgen receptor blocker and the insulin sensitizing agent can be administered at the same frequency or at a different frequency. The androgen receptor blocker and the insulin sensitizing agent can be administered at the same moment or within a certain time interval of each other. The androgen receptor blocker and insulin sensitizing agent can be within the same pharmaceutical carrier but can also be within different carriers. There are different ways to supply the insulin sensitizing agent and androgen receptor blocker under the from of different carriers. They can be in different recipients or within a same recipient. When androgen receptor blocker and insulin sensitizing agent are within the same pharmaceutical carriers, regimens where alternating frequencies for the androgen receptor blocker and insulin sensitizing agent are preferred. Pharmaceutical carriers can be arranged such as in a blister with a weekly or monthly calendar.

Suitable pharmaceutical carriers for use in the pharmaceutical compositions of the invention are described for instance in Remington's Pharmaceutical Sciences 16^{th} ed. (1980) and their formulation is well known to those skilled in the art. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the active ingredients in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredients into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition comprising the active ingredient may require protective coatings. The pharmaceutical form suitable for injection use include sterile aqueous solutions or dispersions and sterile powders for the extemporaneous preparation thereof. Typical carriers therefore include biocompatible aqueous buffers, ethanol, glycerol, propylene glycol, polyethylene glycol and mixtures thereof.

Pharmaceutically acceptable carrier as used herein means any material or substance with which the composition of the active ingredients are formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents may also be prepared by micronisation, for instance in view to obtain them in the form of microspheres usually having a diameter of about 1 to 10 μm, namely for the manufacture of microcapsules for controlled or sustained release of the active ingredients.

Another aspect of the present invention is the reduction in total fat mass after administration of the combination therapy of the present invention. The reduction of fat mass is predominantly a reduction in truncal fat mass and more predominantly a reduction in abdominal fat. A reduction of fat, predominantly abdominal fat is known to reduce the risk of cardiovascular diseases (e.g. Rexrode et al. (1998) ref supra). The waist to hip ratio is used as a parameter to estimate the risk for developing cardiovascular disorders, whereby a WHR of more that 0.76 is indicative of an increased risk for the development of a cardiovascular disorder. The selective reduction in abdominal fat as obtained with the present invention results in a decrease of the waist to hip ratio which is unprecedented with respect to existing therapies for the treatment of PCOS. As shown in the examples, the combination therapy of the present invention succeeds in changing the WHR from values above the critical threshold value 0.76 to values of 0.76 or lower.

With the method and the compositions of the present composition reductions in WHR are obtained of 0.02 or more, preferably 0.04 or more and more preferably 0.04 or more.

An embodiment of the present invention is a method for the reduction of total fat mass. With the method and the compositions of the present invention reductions of total fat mass of 3 percent or more, preferably 5 percent or more, or more preferably 7 percent or more, or even more preferably 10% or more are obtained. This reduction is typically obtained over a period of months, e.g. of six or more months.

An embodiment of the present invention is a method for the reduction of the truncal fat mass. With the method and the compositions of the present invention reductions of truncal fat mass of 5 percent or more, preferably 7.5 percent or more, or more preferably 10 percent or more, or even more preferably 12.5 percent or more are obtained. This reduction is typically obtained over a period of months, e.g. of six or more months.

An embodiment of the present invention is a method for the reduction of the abdominal fat mass. With the method and the compositions of the present invention reductions of abdominal fat mass of 5 percent or more, preferably 10 percent or more, or more preferably 15 percent or more, or even more preferably 20 percent or more are obtained. This reduction is typically obtained over a period of months, e.g. of six or more months.

Another embodiment of the present invention is a method for the increase in lean mass of the body and is indicative of a conversion of fat into muscle. With the method and the compositions of the present composition an increase of the lean mass of the body of 1 percent or more, preferably 2 percent or more, more preferably 3 percent or more, or even 5 percent or more are obtained. This reduction is typically obtained over a period of months, e.g. of six or more months.

Another embodiment of the present invention relates to the change in body shape obtained by the combination therapy of the present invention. This change in body shape is caused by a preferred reduction in abdominal fat. In a more preferred embodiment this change in body shape occurs with no or limited change in body weight. With the method and the compositions of the present invention reductions in total fat and/or truncal fat and/or abdominal fat are obtained whereby the decrease in total body weight is not more than 2 percent, preferably not more than 1 percent and more preferably not more than 0.5 percent. This reduction is typically obtained over a period of months, e.g. of six or more months.

An advantage and unexpected effect of the invention is the more than additive effect of the combination therapy of the present invention compared to monotherapy. The synergistic effects of flutamide and metformin were found to virtually normalize dyslipidemia and circulating androgen levels, indicating that hyperinsulinism and hyperandrogenism each contribute to these endocrine-metabolic abnormalities.

With the compositions and combination therapy of the present invention a more than additive effect is obtained compared to existing monotherapies with respect to the reduction of circulating levels of androstenedione, DHEAS and testosterone. An aspect of the invention is the reduction in levels of androstenedione by at least 20 percent, preferably by at least 30 percent, reduction in levels of DHEAS by at least 25 percent, preferably by at least 35 percent, and reduction in levels of testosterone by at least 30 percent, preferably by at least 40 percent.

Another embodiment of the invention is the synergistic effect of the combination therapy of the present invention compared to monotherapy.

The synergistic impact of flutamide-metformin on serum HDL-cholesterol is of significant clinical importance. In line with previous experience in non-obese (Ibáñez et al. (2000) J Clin Endocrinol Metab. 85:3251-3255.) and obese women with hyperinsulinism-hyperandrogenism (Diamanti-Kandarakis et al. (1998) J Clin Endocrinol Metab. 83:2699-2705.), flutamide was confirmed not to alter serum HDL levels, thus pointing to a lack of endogenous androgen effect on HDL in hyperinsulinemic women. Similarly, metformin in monotherapy was confirmed to increase serum HDL-cholesterol, thus pointing to a HDL-cholesterol-suppressing effect of hyperinsulinism in hyperandrogenic women (Ibáñez et al. (2000). J Clin Endocrinol Metab. 85:3526-3530.). Hence, the synergism of flutamide and metformin in augmenting serum HDL-cholesterol unmasks that the contribution of endogenous androgens to the reduction of HDL-cholesterol in these women depends on the degree of hyperinsulinism. More in general, the pathophysiological principle that effects of hyperandrogenism can be masked by increasing hyperinsulinemia—and possibly vice-versa—may explain some of the apparent inconsistencies in previously reported relationships between androgen—and insulin effects in women (Sozen and Arici (2000) in Obstet Gyn Survey. 55:321-328.; Porettsky et al. (1999) Endocr Rev. 20:535-582.).

It is an aspect of the invention to use the compositions and the combination therapy of the present invention to reduce the levels of circulating triglycerides and LDL cholesterol levels and to increase the levels of circulating HDL-cholesterol.

It is an aspect of the invention to reduce the circulating triglycerides by at least 35 percent, preferably with at least 45 percent, and/or to reduce the levels of LDL cholesterol and/or to increase the levels of HDL-cholesterol by reducing the ratio of LDL cholesterol\HDL cholesterol by at least 35 percent, preferably at least 45 percent.

The present invention will be demonstrated in more detail in the following examples which are however not intended to be limiting the scope of the invention, the latter being only defined by the appended claims.

### EXAMPLE 1

### Comparison of Monotherapy with Flutamide (250 mg/day) or Metformin (1275 mg/day Versus Flutamide/Metformin Combination Therapy (250 mg/day) and Metformin (1275 mg/day) After 9 Months of Therapy

Subjects and Methods

Study Population

The study population consisted of 31 women (age 18.7±0.3 year; range, 18-22 year) who were 5-10 year beyond menarche. At inclusion, each woman had ovarian hyperandrogenism, as defined by amenorrhea or oligomenorrhea (duration of menstrual cycles>45 days); and/or hirsutism (score≧8 on the Ferriman and Gallwey scale) (Ferriman and Gallwey (1961) J Clin Endocrinol Metab. 21:1440-1447.); and elevated serum androstenedione, total testosterone and/or free androgen index [testosterone×100/sex hormone-binding globulin (SHBG), an index of free testosterone (Ibáñfez et al. (1994) J Clin Endocrinol Metab. 79:1778-1784.)]; and a 17-hydroxyprogesterone (17-OHP) hyperresponse (>160 ng/dL) to leuprolide acetate, a GnRH agonist (Procrin, Abbott, Madrid, Spain) (Ibáñez et al. (1994) J Clin Endocrinol Metab. 79:1778-1784; Ibáñez at al. (1993) J Clin Endocrinol Metab. 76:1599-1603.). None of the women had a body mass index (BMI)>25 Kg/m² or had thyroid dysfunction, hyperprolactinemia, a family or personal history of diabetes mellitus, late-onset congenital adrenal hyperplasia (New et al. (1983) J Clin Endocrinol Metab. 56:320-325; Sakkal-Alkaddour et al. (1996) J Clin Endocrinol Metab. 81:3961-3965.), or was receiving a medication known to affect gonadal or adrenal function, or carbohydrate- or lipid-metabolism.

Study Design

At start of the study, the women were considered to be in a steady state condition and they were randomized to receive once daily flutamide [n=10; Eulexin, Schering-Plough Corp., Madrid, Spain, 250 mg], metformin (n=8; Dianben, Andreu-Roche, Barcelona, Spain, 1275 mg] or flutamide-metformin [n=13; Eulexin 250 mg and Dianben 1275 mg] for 6 months.

Before start of treatment, women were screened for blood count, serum electrolytes, lipids and dehydroepiandrosterone-sulfate (DHEAS), and liver and kidney function; a standard 2-h oral glucose tolerance test (oGTT, starting at 0800 am) was performed after three days on a high carbohydrate diet (300 g/day) and an overnight fast. Blood was sampled at 0, 30, 60 and 120 minutes after oral glucose intake, for measurement of glucose and immunoreactive insulin, as described (Ibáñez et al. (1997) J Clin Endocrinol Metab. 82:2283-2288; Ibáñez et al. (1999) J Clin Endocrinol Metab. 84: 2691-2695). During the oGTT, the areas under the curves for glucose (mean serum glucose) and insulin (MSI) were calculated according to the trapezoidal rule. At start of treatment, all women had normal glucose tolerance (The Expert Committee on the Diagnosis and Classification of Diabetes Mellitus. in (1997) Report of the Expert Committee on the Diagnosis and Classification of Diabetes Mellitus. Diabetes Care. 20:1183-1197), but were hyperinsulinemic, as judged by peak serum insulin concentrations>150 μU/mL during oGTT (Vidal-Puig and Moller (1997) In Azziz et al. Androgen excess disorders in women. Philadelphia: Lippincott-Raven Publishers; p 227-236.) and/or MSI values>84 mU/L (Ibáñez et al. (1997) J Clin Endocrinol Metab. 82:2283-2288; Ibáñez et al. (1999) J Clin Endocrinol Metab. 84: 2691-2695.).

After 6 months on either monotherapy or combined therapy, fasting glucose and insulin were reassessed together with serum LH (luteinizing hormone), FSH (Follicle stimulating hormone), estradiol, testosterone, androstenedione, DHEAS, SHBG, and lipid profile. Blood count and liver-kidney function were also screened after 1, 3, and 6 months, as additional safety variables.

Ovulation Assessment

Prior to start of treatment, ovulatory function was documented twice (months −3 and −1) by measuring serum progesterone concentrations in 4 consecutive samples obtained with an interval of 1 week. Ovulation rate was assessed similarly after 2, 4 and 6 months on treatment; ovulation was post-factum considered to have occurred if a serum progesterone level>8 ng/mL was found in a sample obtained 5-8 days before menses (Nestler et al. (1998) N Eng J Med 338:1876-1880.). The ovulation results before start of treatment are the average of the results obtained during month -3 and -1, whereas the 3 months results are the mean of those obtained after 2 and 4 months on treatment.

Hormonal Assays, Statistics & Ethics

Serum glucose was measured by the glucose oxidase method. Immunoreactive insulin was assayed by IMX (Abbott Diagnostics, Santa Clara, Calif.). The mean intra-and inter-assay coefficients of variation (CVs) were 4.7% and 7.2%, respectively. LH, FSH and progesterone were measured by immuno-chemiluminiscence (IMMULITE 2000, Diagnostic Products Corp, Los Angeles, Calif.), with CVs of 3.5% and 5.0% for LH, 4.6% and 6.3% for FSH, and 7.8% and 8.5% for progesterone. Serum testosterone, 17-OHP, androstenedione, estradiol, SHBG and DHEAS levels were assayed as previously described (Ibáñez et al. (2000) J Clin Endocrinol Metab. 85:3526-3530.). Serum samples were kept frozen at −20° C. until assay.

Anthropometric data and hormonal results are expressed as mean, unless stated otherwise. Comparisons were made by X-square or t-test, p-values≦0.01 being considered as statistically significant.

Informed consent was obtained from each woman. The study design was approved by the Institutional Review Board of Barcelona Hospital; in view of available evidence (Ibáñez et al. (2000) J Clin Endocrinol Metab 85:3251-3255., Ibáñez et al. (2000) J Clin Endocrinol Metab. 85:3526-3530.), inclusion of a randomized, untreated control group was judged ethically unacceptable.

Results

Table 1 and the figure summarize the clinical, biochemical and endocrine-metabolic characteristics of the study population and the randomized sub-populations before and after 9 months on either monotherapy or combined flutamide-metformin treatment

TABLE 1
clinical biochemical and endocrine metabolic parameters of flutamide
metformin monotherapy and combined therapy after 9 months

| | | | | Flutamide |
|---|---|---|---|---|
| | | | | (250 |
| | | | | mg/day) + |
| | | Flutamide | Metformin | Metformin |
| | | (250 | (1275 | (1275 |
| | Reference | md/day) | mg/day) | mg/day) |
| | value | n = 10 | n = 8 | n = 13 |
| Age (yr) | | 18.8 | 18.6 | 19.8 |
| Body mass | 21.6 | 22.3 | 22.5 | 21.2 |
| index | | | | |
| (Kg/m²) | | | | |
| Ferriman & | < | 14.1 | 18.0 | 16.2 |
| Gallwey | | | | |
| score | | | | |
| LH (IU/L) | 5.1 | 8.8 | 7.2 | 8.6 |
| FSH (IU/L) | 5.4 | 4.6 | 4.9 | 5.1 |
| Estradiol | 28 | 64 | 57 | 63 |
| (pg/mL) | | | | |
| Testos- | 31 | 108 | 132 | 104 |
| terone | | | | |
| (ng/dL) | | | | |
| SHBG | 1.9 | 0.8 | 0.7 | 0.9 |
| (μg/dL) | | | | |
| Androstene- | 126 | 272 | 274 | 336 |
| dione | | | | |
| (ng/dL) | | | | |
| DHEAS | 133 | 231 | 264 | 301 |
| (μg/dL) | | | | |
| Peak 17- | 93 | 216 | 253 | 260 |
| OHP | | | | |
| Glucose | 80 | 83 | 83 | 85 |
| (mg/dL) | | | | |
| Insulin | 11.3 | 14.4 | 14.9 | 11.7 |
| (mU/L) | | | | |
| Insulin | 77.0 | 69.9 | 70.5 | 80.9 |
| Sensitivity | | | | |
| (HOMA %) | | | | |
| Total | 146 | 182 | 190 | 187 |
| cholesterol | | | | |
| (mg/dL) | | | | |
| LDL-choles- | 70 | 106 | 111 | 116 |
| terol | | | | |
| (mg/dL) | | | | |
| HDL-choles- | 62 | 55 | 59 | 51 |
| terol | | | | |
| (mg/dL) | | | | |
| Trigly- | 61 | 94 | 84 | 109 |
| cerides | | | | |
| (mg/dL) | | | | |

SHBG, sex hormone-binding globulin;
DHEAS, dehydroepiandrosterone-sulfate
peak 17-OHP, peak 17-hydroxyprogesterone after GnRH agonist

After 9 months on treatment, significant differences were apparent between the treatment groups (Table 1). The women who received combined flutamide-metformin therapy show less hirsutism, lower levels of testosterone, androstenedione, DHEAS and LDL-cholesterol, and higher SHBG levels and insulin sensitivity than other groups. Women who received metformin alone, or in combination with flutamide, have higher HDL-cholesterol and lower triglyceride levels than women who received flutamide only. Comparisons of percentage change from baseline showed that compared to flutamide alone, combined flutamide-metformin therapy resulted in significantly greater improvements in insulin sensitivity, LDL/HDL-cholesterol ratio and triglyceride, testosterone, androstenedione and DHEAS levels (Figure); compared to metformin alone, combined flutamide-metformin therapy resulted in significantly greater improvements in LDL/HDL-cholesterol ratio and triglyceride, androstenedione and DHEAS levels (Figure). No treatment produced any change in BMI (post-treatment vs pre-treatment: p>0.8 for all treatment groups, Table 1).

The monthly fraction of ovulatory cycles, as monitored by weekly serum progesterone measurements, increased with metformin alone (baseline vs. after 9 months: 5% vs 75%, p<0.01) and also with combined therapy (8% vs 92%, p<0.0001), but were unimproved with flutamide alone (15% vs 20%, p=NS).

Each treatment regimen was well tolerated; indices of hepatic and renal function were stable throughout the study. One woman receiving flutamide (1 of 10) complained of dry skin; three women receiving metformin or flutamide-metformin (3 of 21) reported transient abdominal discomfort.

### EXAMPLE 2

### Comparison of Monotherapy with Flutamide (125 mg/day) or Metformin (1275 mg/day) Versus Flutamide/Metformin Combination Therapy (250 mg/day) and Metformin (1275 mg/day) After 9 Months of Therapy

Study Population

The study population consisted of 30 adolescent girls (age, 15.8+0.3 yr; range, 13.6-18.6 yr), who were 3-8 yr beyond menarche and who were either not at risk of pregnancy or using a non-hormonal contraceptive method. The inclusion criteria for this cohort were identical to those describes in example 1.

Study Design

At start of this open-labeled study, the girls were considered to be in a steady state condition. The study population (n=30) was randomized for an onset of treatment that was either delayed by 3 months (n=14; controls) or not delayed (n=16, treated); all girls received flutamide-metformin (Flutamide, Merck Farma y Química, Barcelona, Spain, 125 mg and Dianben, Andreu-Roche, Barcelona, Spain, 1275 mg, once daily) for a period of 6 months.

Waist to Hip Ratio and Body Composition

Waist circumference was measured at the end of expiration to the nearest millimetre, using a measuring tape placed around the waist at the level of the umbilicus. Hip circumference was also measured to the nearest millimetre, at the level of maximal anteroposterior hip excursion. Waist and hips were each measured three times, and the mean of each measurement was used for analyses; waist to hip ratios were also calculated.

Body composition was assessed by dual-energy x-ray absorptiometry (DXA) using a Lunar Prodigy machine. All studies were performed using Lunar software programs (versions 3.4 and 3.5, Lunar Corp., Madison, Wis., USA) (Ibáñez et al. (2000) Horm Res. 54:192-197). Absolute (Kg) and relative (percentage, %) whole body fat and lean mass, as well as fat content (Kg and %) of specific regions of the body (trunk and abdomen) were assessed. The truncal region was defined as the tissue area bordered by a horizontal line below the chin, vertical borders lateral to the ribs, and oblique lines passing through the femoral necks. The abdominal region was defined as the area encompassed between the dome of the diaphragm (cephalad limit) and the top of the greater throcanter (caudal limit) (Taylor et al. (1998) Am J Clin Nutr. 67:44-49.). The total radiation dose in each examination was 0.1 m Sievert. The coefficients of variation for scanning precision, calculated from 30 consecutive scans of an external hydroxyapatite, luciate and high-density polyetylene Hologic phantom (Hologic Inc., Waltham, Mass., USA), were 2.0%, and 2.6%, respectively, for fat and lean body mass (Kiebzak et al. (2000) J Clin Densitometry. 3:35-41.). The CVs for abdominal fat mass completed on 3 consecutive scans from 14 subjects was 0.74%.

Waist and hip circumferences, waist-to-hip ratios and body composition were documented before and after 6 months on treatment. Waist and hip circumferences and waist-to-hip ratios were also assessed after 3 months on treatment.

Hormonal Assays, Statistics and Ethics

Fasting glucose, insulin, LH, FSH, estradiol, testosterone, androstenedione, dehydroepiandrosterone-sulfate (DHEAS), SHBG, and lipid profile were measured at baseline and subsequently at three-month intervals until the completion of the 6-months treatment period.

Blood count and liver and kidney function tests were also screened after 1, 3 and 6 months on treatment, as additional safety variables.

Ovulation Assessment

Prior to start of treatment, ovulatory function was documented by measuring serum progesterone concentrations in 4 consecutive weekly samples. Ovulation rate was assessed similarly after 6 months on treatment; ovulation was post-factum considered to have occurred if a serum progesterone level>8 ng/mL was found in a sample obtained 5-8 days before menses (Nestler et al. (1998) N Eng J Med. 338:1876-1880.).

Hormonal, biochemical and metabole-endocrine assays were performed as described in Example 1. Anthropometric data and hormonal results are expressed as mean, unless stated otherwise. Comparisons were made by two sided t-test, p values<0.05 being considered as statistically significant. The study was conducted in Barcelona after approval by the Institutional Review Board of Barcelona Hospital; informed consent was obtained from parents and/or girls, as well as assent from minors.

Table 2 summarizes the clinical characteristics, as well as the endocrine-metabolic status and ovulation rates of the adolescents before and after 3 and 6 months on combined flutamide-metformin therapy. In untreated girls, all study indices remained stable during the control phase between−3 and 0 months. At start of treatment (0 months), all clinical, biochemical and endocrine-metabolic variables were comparable in the two subgroups receiving immediate or delayed treatment and, hence, these subgroups were pooled for subsequent, paired analyses.

Combined flutamide-metformin treatment was accompanied by marked decreases in hirsutism score, fasting insulin, free androgen index, total testosterone, androstenedione and DHEAS, by an increase in serum SHBG, and by a less atherogenic lipid profile (all p<0.0001). Most changes were already evident after the first 3 months on combined therapy.

Eumenorrhea was restored in 29/30 girls, and monthly ovulation rates rose from 14% to 67% within 6 months.

The correction of endocrine-metabolic abnormalities was accompanied by a striking decrease in waist-to-hip ratio attributable to a reduction in waist circumference, by a marked reduction in truncal and abdominal fat mass, and by an increase in lean body mass (all p<0.0001) without detectable change in total body weight.

TABLE 2
Clinical, hormonal and dexa variables before treatment and on flutamide-
metformin treatment for 3-6 months.

| | | Pre-treatment | | | | Treatment | |
|---|---|---|---|---|---|---|---|
| | | control | control | treated | total | Total | Total |
| | | −3 mo | 0 mo^{$} | 0 mo | 0 mo | 3 mo | 6 mo |
| | reference | (n = 14) | (n = 14) | (n = 16) | (n = 30) | (n = 30) | (n = 30) |
| Body mass index | 21.6 | 21.8 | 21.7 | 21.7 | 21.7 | 21.6 | 21.4 |
| (Kg/m²) | | | | | | | |
| Ferriman & Gallwey | <8 | 15.4 | 15.5 | 14.4 | 14.9 | 12.9^{¶} | 11.1^{#} |
| score | | | | | | | |
| Waist circumference | 66.6 | 73.0 | 74.2 | 71.6 | 72.8 | 70.2^{¶} | 69.4^{#} |
| (cm) | | | | | | | |
| Waist-to-hip ratio | 0.741 | 0.780 | 0.785 | 0.766 | 0.777 | 752^{¶} | 738^{#} |
| Ovulatory: | | 1:13 | | 1:15 | 2:28 | | 20:10\hat{ } |
| Anovulatory | | | | | | | |
| LH (IU/L) | 5.1 | 5.8 | 6.8 | 7.7 | 7.2 | 5.7 0.6 | 6.7 |
| FSH (IU/L) | 5.4 | 6.2 | 5.0 | 4.8 | 4.9 | 4.9 0.3 | 4.7 |
| Glucose (mg/dL) | 80 | 87 | 86 | 90 | 88 | 83.1^{‡} | 84 |
| Insulin (mU/L) | 11.3 | 15.8 | 14.9 | 16.0 | 15.5 | 10.5^{¶} | 9.7 |
| SHBG (μg/dL) | 1.9 | 1.0 | 1.0 | 0.9 | 0.9 | 1.1^{‡} | 1.1 |
| Testosterone (ng/dL) | 31 | 116 | 126 | 136 | 132 | 84^{¶} | 69^{#} |
| Androstenedione | 126 | 264 | 271 | 296 | 285 | 232^{¶} | 225 |
| (ng/dL) | | | | | | | |
| DHEAS (μg/dL) | 133 | 238 | 247 | 255 | 251 | 202^{¶} | 181* |
| LDL-cholesterol | 70 | 111 | 115 | 110 | 112 | 85^{¶} | 77* |
| (mg/dL) | | | | | | | |
| HDL-cholesterol | 62 | 52 | 51 | 54 | 52 | 63^{¶} | 64 |
| (mg/dL) | | | | | | | |
| Triglycerides (mg/dL) | 61 | 70 | 79 | 79 | 79 | 55^{¶} | 53 |
| BMD (gr/cm²) | 1.00 | 1.10 | 1.12 | 1.16 | 1.15 | | 1.14 |
| BMC (g) | 1.75 | 2.18 | 2.21 | 2.26 | 2.24 | | 2.23 |
| Total fat mass (Kg) | 12.9 | 18.3 | 19.0 | 18.8 | 18.9 | | 17.6^{¶} |
| Truncal fat mass (Kg) | 5.4 | 8.6 | 9.0 | 8.9 | 9.0 | | 8.2^{¶} |
| Abdominal fat mass | 3.0 | 5.2 | 5.5 | 5.7 | 5.6 | | 4.9^{¶} |
| (Kg) | | | | | | | |
| Lean body mass (Kg) | 31.6 | 35.8 | 35.4 | 35.4 | 35.4 | | 36.4^{¶} |

Values are mean
SHBG, sex hormone-binding globulin;
DHEAS, dehydroepiandrosterone-sulfate;
BMD, bone mineral density;
BMC, bone mineral content.
^{$}p = NS for all variables versus −3 months
^{‡}p < 0.01;
^{¶}p < 0.0001 versus 0 months
*p < 0.01;
^{#}p < 0.0001 versus 3 months

### EXAMPLE 3

### Changes in Endocrine-Metabole Values and Body Composition After Flutamide (125/mg day) Metformin (1275 mg/day) Combination Therapy After 9 Months

The study population consisted of 30 adolescent girls ((n=30), age 15.8+1.7 yr, range 13.3-19 yr) who were 5-6 years beyond menarche and who were neither at risk of pregnancy or using a non-hormonal contraceptive method. Inclusion criteria, study design, ovulation assessment, hormonal assays, body composition measurements and analysis of the results was performed as in examples 1 and 2.

In this study the dose of flutamide was lowered with 50% to a daily dose of 125 mg/day while the dose of metformin was not changed (1275 mg/day) Table 3 summarizes relevant clinical characteristics, as well as the endocrine-metabolic status rates of the adolescents before and after 3 and 6 months on combined flutamide-metformin therapy. In untreated girls, all study indices remained stable during the control phase between −3 and 0 months. At start of treatment (0 months), all clinical, biochemical and endocrine-metabolic variables were comparable in the two subgroups receiving immediate or delayed treatment and, hence, these subgroups were pooled for subsequent, paired analyses.

Combined flutamide-metformin treatment was accompanied by marked decreases in hirsutism score (mean value from 14.9 to 10.1 after 9 months), fasting insulin, free androgen index, total testosterone, androstenedione and DHEAS, by an increase in serum SHBG (mean value form 31.6 to 39.7 after nine months), and by a less atherogenic lipid profile (all p<0.0001). Most changes were already evident after the first 3 months on combined therapy.

Eumenorrhea was restored in 29/30 girls after 6 monts, and monthly ovulation rates rose from 14% to 67% within 6 months.

The correction of endocrine-metabolic abnormalities was accompanied by a striking decrease in waist-to-hip ratio attributable to a reduction in waist circumference, by a marked reduction in truncal and abdominal fat mass, and by an increase in lean body mass (all p<0.0001) without detectable change in total body weight.

TABLE 4
changes in biochemical data for glycerides and
hormones and changes in body composition
after combination therapy with reduced levels
of flutamide (125 mg/day)

| | | | waist | total fat | truncal | abdominal | | |
|---|---|---|---|---|---|---|---|---|
| | LDL/ | DEAS | to hip | mass | fat mass | fat mass | lean body | total |
| months | HDL | microg/dL | ratio | (kg) | (kg) | (kg) | mass (kg) | weigth |
| −3 | nd | nd | 0.78 | 18.6 | 8.8 | 5.3 | 35.7 | 54.0 |
| 0 | 2.2 | 251 | 0.78 | 18.9 | 9 | 5.6 | 35.3 | 53.9 |
| +3 | 1.4 | 202 | 0.75 | nd | nd | nd | nd | 53.6 |
| 6 | 1.2 | 180 | 0.74 | 17.5 | 8.1 | 4.9 | 36.4 | 53.3 |
| 9 | 1.2 | 180 | 0.73 | 16.5 | 7.7 | 4.3 | 37.3 | 53.3 |
| Δ 0-6 | −27% | −28% | 0.04 | −7.4% | −10.0% | −12.5% | +3.4% | −0.5% |
| Δ 0-9 | −27% | −28% | 0.05 | −12.5% | −14.4% | −23.2% | +5.6% | −0.5% |

values are mean values, n = 30
Δ 0-6 and Δ 0-9 shows changes of the mean values compared to the start of the study (o months) after 6 and 9 months.

### EXAMPLE 4

### Changes in Endocrine-Metabole Values and Body Composition After Flutamide (62.5/mg day)-Metformin (1275 mg/day) Combination Therapy After 3 Months

A next study was performed to study the effect of even lower doses of flutamide (62.5 mg/day) was performed. At the moment of filing a limited number of persons was treated and data were collected for a period of three months.

Data are presented from 5 adolescent girls between 14 and 18 who were between 3 to 7 year beyond menarche and were either not at risk of pregnancy or using a non-hormonal contraceptive method.

Inclusion criteria, study design, treatment, ovulation assessment, hormonal assays, body composition measurements and analysis of the results was performed as in examples 1 and 2, with daily doses of flutamide of 62.5 mg and daily doses of metformin of 1275 mg.

A control group of 13 girls, aged between 12.6 and 16 years who were between 3 to 5 years beyond menarche, received no treatment.

TABLE 4
changes in biochemical data for glycerides and hormones and changes in body
composition after combination therapy with reduced levels of flutamide (62.5 mg/day)

| | | | waist | | | abdominal | lean | |
|---|---|---|---|---|---|---|---|---|
| | | | to | total fat | truncal | fat | body | |
| | | DEAS | hip | mass | fat mass | mass | mass | |
| | LDL/HDL | μg/dL | ratio | (kg) | (kg) | (kg) | (kg) | weight |
| control | | | | | | | | |
| 0 months | 1.7 | 255 | 0.764 | 14.9 | 6.7 | 3.54 | 34.6 | 50.1 |
| 3 months | 1.5 | 194 | 0.761 | 15.8 | 7.2 | 4.0 | 34.4 | 50.5 |
| Δ 0-3 | −10% | −24% | −0.003 | +6.0% | +7.5% | +13.4% | −0.6% | +0.8% |
| test | | | | | | | | |
| cohort | | | | | | | | |
| 0 months | 1.7 | 225 | 0.783 | 19.0 | 9.1 | 5.0 | 39.4 | 57.6 |
| 3 months | 1.5 | 182 | 0.773 | 19.3 | 9.2 | 3.9 | 39.5 | 59.7 |
| Δ 0-3 | −10% | −20% | −0.010 | +1.1% | +1.1% | −24% | +1.1% | +2.1% |

values are mean, n = 11 for the control population, n = 5 for the test cohort

Within this short time period a considerable change is already noticed in the reduction in waist to hip ratio. In the control groups the lean mass is decreasing, the test cohort shows an increase in lean mass. Significant changes are noticed after three months in the fat composition. As well total fat, truncal fat and abdominal fat are increasing in the control population. The group treated with flutamide metformin shows a dramatic decrease in abdominal fat mass which is also reflected in the values for abdominal fat and truncal fat which only slightly increase in the test population. The conversion of fat to muscle is reflected by the increase in lean body mass.

### EXAMPLE 5

### Flutamide-Metformin Combination Therapy Supplemented With Contraceptives

31 non-obese, young women with hyperinsulinemic hyperandrogenism were treated with flutamide (Flu, 250 mg/d) and metformin (Met, 1275 mg/d) for 9 months. After 9 months, all women received Flu-Met, but with a lower dose of flutamide (125 mg/d); between 12-18 months, a low-dose, monophasic, estro-progestogen OC (ethinylestradiol 20 mcg+gestodene 75 mcg; Meliane, Schering) was added, in case of pregnancy risk. A total of 12 young women elected to add an OC (OC+), and this subgroup was then matched to a subgroup of 12 women who continued on Flu-Met alone (OC−); clinical and endocrine-metabolic variables were matched, both at 0 months and after 12 months (Table 5).

Comparisons between the endocrine-metabolic results before vs on Flu-Met (0 vs 12 months) show the broad and major impact of such combined treatment, while 12 vs 18 months comparisons within the OC− subgroup show that the newly induced equilibrium remains stable over those 6 months; the novel equilibrium is also maintained in the OC+ subgroup, except for the anticipated increment in SHBG and the ensuing drop in free androgen index.

There is a solid pathophysiological basis to consider a low-dose combination of an androgen receptor blocker and an insulin-sensitizer as a therapeutic approach for women with hyperinsulinemic hyperandrogenism. However, since an increment of ovulation rate is part of the remarkable efficacy of this combination, the timely addition of contraceptive measures is a major point of attention. The present findings indicate that the main clinical and endocrine-metabolic benefits of a low-dose Flu-Met combination are maintained after addition of a low-dose estro-progestagen.

TABLE 5
Endocrine-metabolic variables, in fasting condition, at study start
(0 months) and on flutamide-metformin (FluMet; 12 months); thereafter
(12-18 months), an oral contraceptive (OC) consisting of a low-dose
estro-progestagen (Meliane^{R}) was either added to flutamide-metformin
(FluMet/OC+) or not (FluMet/OC−) for 6 months.

| | 0 months# | | 12 months | | 18 months | |
|---|---|---|---|---|---|---|
| | (FluMet/ | (FluMet/ | (FluMet | FluMet | FluMet/ | FluMet/ |
| | OC−) | OC+) | OC−) | (OC+) | OC− | OC+ |
| Glucose | 84 | 83 | 84 | 82 | 84 | 86 |
| (mg/dL) | | | | | | |
| Insulin (mU/L) | 14.1 | 12.1 | 10.6^{&} | 9.7 | 9.9 | 9.3 |
| SHBG (μg/dL) | 0.8 | 0.8 | 1.3^{&} | 1.4^{&} | 1.3 | 2.60^{φ} |
| Testosterone (ng/dL) | 127 | 102 | 69^{&} | 62^{&} | 69 | 72 |
| Free androgen | 17.6 | 12.9 | 5.8^{&} | 4.8^{&} | 5.4 | 2.8^{φ} |
| index^{§} | | | | | | |
| Androstenedione (ng/dL) | 312 | 274 | 221^{&} | 186^{&} | 213 | 204 |
| DHEAS | 275 | 240 | 205^{&} | 167^{&} | 187 | 152 |
| (μg/dL) | | | | | | |
| LDL- | 111 | 110 | 76^{&} | 79^{&} | 77 | 85 |
| cholesterol | | | | | | |
| (mg/dL) | | | | | | |
| HDL- | 56 | 55 | 69^{&} | 68^{&} | 69 | 70 |
| cholesterol | | | | | | |
| (mg/dL) | | | | | | |
| Triglycerides (mg/dL) | 95 | 92 | 58^{&} | 59^{&} | 50 | 71 |
| Ovulatory:Anovulatory | 2:10 | 1:11 | 8:4^{&&} | 9:3^{&&} | — | — |

Values are mean
p-values by two-sided t-test.
^{#}p > 0.01 between OC− and OC+ subgroups, for all variables.
*For treatment between 0-9 months: see Table 1; treatment between 9-12 months: Flutamide (125 mg/d) + Metformin (1275 mg/d).
^{&}p ≦ 0.01 vs start (^{&&}by Chi-square);
^{φ}p < 0.01 vs 12 months.
SHBG, sex hormone-binding globulin;
DHEAS, dehydroepiandrosterone-sulfate;
^{§}Testosterone (nmol/L) × 100/SHBG (nmol/L)

1. A composition, for simultaneous or sequential application, comprising an androgen receptor blocker and a insulin sensitizing agent.

2. The composition of claim 1 wherein the androgen receptor blocker is flutamide a derivative, analog or salt thereof.

3. The composition of claim 1 or 2 wherein the insulin sensitizing agent is metformin a derivative, analog or salt therefor.

4. The composition according to any previous claim further comprising an contraceptive.

5. The composition according to any of the claims 2 to 4 wherein the concentration of flutamide is between about 50 and about 250 mg.

6. The composition according to any of claims 3 to 5 wherein concentration of metformin is between about 400 and about 1700 mg.

7. The composition according to any of claims 1 to 6 wherein the androgen receptor blocker has a concentration equivalent to an amount of flutamide in the concentration range of about 50 to about 250 mg flutamide as determined by an assay such as an androgen promoter reporter assay.

8. The composition according to any of claims 1 to 7 wherein the insulin sensitizing agent has a concentration equivalent to an amount of metformin in the concentration range of about 400 to about 1700 mg metformin as determined by an assay such as an assay which comprises the step of determining the decrease in circulating insulin in the blood after administration of an insulin sensitizing agent.

9. The composition according to any previous claim wherein the androgen receptor blocker and the insulin sensitizing compound are within the same pharmaceutical carrier.

10. The composition according to any of the claims 1 to 8 wherein the androgen receptor blocker and the insulin sensitizing compound are within different pharmaceutical carriers.

11. A method for reducing the total amount of fat in a female human person suffering from polycystic ovary syndrome comprising administering to said human subject an effective amount of an androgen receptor blocker and an insulin sensitizing compound, either sequentially or simultaneously.

12. The method of claim 11 wherein the reduction in total amount fat is accompagnied by a decrease of the total amount of fat by at least 3%.

13. The method of claim 11 wherein the reduction in total amount of fat is accompanied by a decrease of truncal fat by at least 5%.

14. The method of claim 11 wherein reduction in total amount of fat is accompanied by a decrease of abdominal fat by at least 6%.

15. The method of claim 11 wherein reduction in total amount of fat is accompanied by an increase of lean mass by at least 1%.

16. The method of claim 11 wherein reduction in total amount of fat is accompanied by a decrease in waist to hip ratio from a value above 0.76 to a value of 0.76 or below.

17. The method of claim 11 wherein the reduction in total amount of fat is accompanied by a decrease in waist to hip ratio with a value of at least 0.02.

18. The method of claim 11 wherein reduction in total amount of fat is accompanied by a decrease of total body weight of not more than 2 percent.

19. A method for treating a female human person suffering from polycystic ovary syndrome comprising administering to the human subject an effective amount of an androgen receptor blocker and an insulin sensitizing compound either sequentially or simultaneously.

20. The method of claim 19 wherein the levels of androstenedione are reduced by at least 30 percent.

21. The method of claim 19 wherein the levels of DHEAS are reduced by at least 30 percent.

22. The method of claim 19 wherein the levels of testosterone are reduced by at least 30 percent.

23. The method of claim 19 wherein the levels of circulating triglycerides are reduced by at least 35 percent.

24. The method of claim 19 wherein the of ratio of LDL cholesterol/HDL cholesterol is reduced by at least 35 percent.

25. The method of any of claims 1 1 to 24 wherein the androgen receptor blocker is flutamide or a derivative, salt or analog thereof.

26. The method of any of claims 11 to 25 wherein the concentration of flutamide is about 250 mg.

27. The method of any of claims 11 to 24 wherein the concentration of flutamide is between about 250 mg and about 125 mg.

28. The method of any of claims 11 to 24 wherein the concentration of flutamide is about 250 mg.

29. The method of any of claims 11 to 24 wherein the concentration of flutamide is between about 125 mg and about 50 mg.

30. The method of any of claims 11 to 24 wherein the concentration of flutamide is about 50 mg.

31. The method of any of claims 11 to 24 wherein the androgen receptor blocker has a concentration equivalent to an amount of flutamide in the concentration range of about 50 to about 250 mg flutamide as determined by an assay such as an androgen responsive element reporter assay.

32. The method of any of claims 11 to 24 wherein the insulin sensitizing compound is metformin or a derivative, analog or salt thereof.

33. The method according to any of claims 11 to 24 wherein the concentration of metformin is between about 800 and about 1700 mg.

34. The method according to any of claims 11 to 24 wherein the concentration of metformin is between about 400 and about 800 mg.

35. The method according to any of claims 11 to 24 wherein the insulin sensitizing agent has a concentration equivalent to an amount of metformin in the concentration range of about 400 to about 1700 mg metformin as determined by an assay such as an assay which comprises the step of determining the decrease in circulating insulin in the blood after administration of an insulin sensitizing agent.

36. Use according to any of claims 11 to 35 further comprising an contraceptive.

37. Use according to claim 36 wherein the contraceptive is an oestroprogestagen.
